(19) United States
(12) Patent Application Publication
(10) Pub. No.: US 2011/0034990 A1
(43) Pub. Date: Feb. 10, 2011

(54) BIOCORRODIBLE IMPLANT WITH ACTIVE COATING

(76) Inventor: Alexander Borck, Aurachtal (DE)

Correspondence Address: GREER, BURNS & CRAIN, 300 S WACKER DR, 25TH FLOOR, CHICAGO, IL, 60606, US

(21) Appl. No.: 12/830,773

(22) Filed: Jul. 6, 2010

Related U.S. Application Data
(60) Provisional application No. 61/231,685, filed on Aug. 6, 2009.

(51) Int. Cl. A61F 2/82; B05D 7/00

(52) U.S. Cl. 623/1.15; 623/1.46; 427/2.25

(57) ABSTRACT

One embodiment of the invention concerns an implant with a basic body of biocorrodible, metallic implant material with an active coating and/or cavity filling.

### CROSS REFERENCE

The present application claims priority on U.S. Provisional Application No. 61/231,685 filed on Aug. 6, 2009.

### FIELD

One example embodiment of the invention concerns an implant with a basic body of a biocorrodible, metallic implant material with an active coating and/or cavity filling.

### BACKGROUND

Implants have applications in many embodiments in modern medical technology. They are used, for example, to support vascular structures, hollow organs and endovascular implants for fastening and temporary fixation of tissue implants and tissue transplants, but also for orthopedic purposes, for example, as nails, plates or screws.

Thus, for example, the implantation of stents has established itself as one of the most effective therapeutic steps in the treatment of vascular disease. The purpose of stents is to provide a supporting function in the hollow organs of a patient. Conventionally built stents have a filigree bearing structure of metallic rods that are first present in a compressed form for insertion into the body and are expanded at the application site. One of the primary application areas of such stents is the permanent or temporary expansion of vascular stenosis and maintaining such in open position, particularly stenosis of the coronary blood vessels. In addition, aneurism stents are known, for example, which are used to support damaged vascular walls.

Stents have a circumference wall of sufficient carrying capacity in order to keep the constricted vascular structure open to the desired degree, and the blood flows unimpeded through a tubular basic body. In many cases, the circumference wall is designed as a grate-like bearing structure that makes it possible to insert the stent in compressed condition in which it has a small diameter up to the stenosis of the respective vascular structure that is to be treated and to expand it there to such an extent, for example, with the help of a balloon catheter, that the vascular structure has the desired enlarged interior diameter. The process of positioning and expanding the stent during the procedure, and the final position if the stent in the tissue after completion of the procedure, must be monitored by a cardiologist. This can be done using imaging procedures such as, for example by X-ray examinations.

The implant or stent has a basic body made of an implant material. An implant material is an inorganic material that is used for a medical application and interacts with biological systems. A basic requirement for a material used as implant material which comes in contact with the body is its biocompatibility. Biocompatibility is understood to mean the ability of the material to provoke an appropriate reaction of the tissue in a specific application. This includes the adaptation of the chemical, physical, biological and morphological surface properties of an implant to the recipient tissue with the goal of a clinically desired interaction. The biocompatibility of the implant material is also dependent on the chronological reaction of the biosystem that receives the implant. Thus, irritations and inflammations occur relatively quickly, which could lead to tissue changes. Thus, biological systems react in various ways depending on the properties of the implant material. According to the reaction of the biosystem, the implant materials can be divided into bioactive, bioinert and degradable/resorbable materials.

A biological reaction to polymeric, ceramic or metallic implant materials depends on the concentration, duration of the effect and the type of introduction. Often, the presence of an implant material leads to an inflammatory reaction that can be triggered by mechanical stimuli, chemical substances but also metabolic products. As a rule, the inflammatory process is accompanied by the immigration of neutrophilic granulocytes and monocytes through the vascular walls, the immigration of lymphocyte effectors by building specific antibodies against the inflammation stimulus, the activation of the complementary system by releasing complementary factors which act as mediators and finally, activation of blood coagulation. An immunological reaction is most often closely connected with the inflammatory reaction and can lead to allergization and allergy formation. Known metallic allergens comprise, for example, nickel, chrome and cobalt that are also used in many surgical implants as components of alloys. An important problem in stent implantation into vascular structures is in-stent restenosis because of overshooting neointimal growth, which is caused by the strong proliferation of the arterial smooth muscle cells and causes a chronic inflammation reaction.

A promising method for solving the problem lies in the use of biocorrodible metals and their alloys as implant material, because most often, the stent is not required to provide a permanent support function; the body tissue that was damaged at first regenerates.

It can be a problem when using these biocorrodible implants that they are entirely or partially made of a metallic material, that degradation products that are created in the corrosion process of the implant are created and released, which often have a notable influence on the local pH value and can lead to undesirable tissue reactions. Additionally, these biocorrodible implants, because of their increased rate of corrosion, often have an implant integrity that is too short for the desired purpose of use and the implant site. Particularly in the degradation process of Mg-containing biocorrodible implant materials, an increase in the pH value can occur in the immediate environment. This increase in pH value can lead to a phenomenon that is summarized under the term alkalosis. The local increase in pH value thereby leads to an imbalance in the load distribution of the smooth muscle cells surrounding the vascular structure, which can lead to a local increase in tonicity in the area of the implant. This increased pressure on the implant can lead to the premature loss of the integrity of the implant. If the implant is a stent, for example, in the course of such a vascular constriction in the vascular structure around the stent, a restenosis could occur or an impairment of the vascular lumen.

In order to prevent the risk factors of a restenosis, a number of coatings continued to be developed for stents that are to offer increased hemo-compatibility. However, these coated implants have, as a rule, a short durability, i.e. they can only be stored for a short time or they also require special storage conditions such as, for example, storage of the products at 4° C. This leads to an increased number of rejects of finished products and thus to an increased economic loss.

A further problem in the optimization of stents with active ingredients is the setting of the dosage of the active ingredient that is to be released. Hereby, it is limiting that the quantity of the active ingredient that can be put on the outside of the stent is severely limited, as the surfaces that are available for application are very small. In stents of biocorrodible magnesium alloys, there can be an additional problem that the strongly alkaline environment that is created by the corrosion of the material, the resorption behavior of the active ingredient that is to be absorbed is influenced negatively. Thus, active ingredients are sometimes used as hydrochlorides when the solubility of the active ingredient is otherwise too small. Such hydrochlorides are, however in the alkaline environment that is being created, again converted into the difficult to dissolve deprotonized active ingredients.

### SUMMARY

In some embodiments of the invention, the resistance to corrosion of the biocorrodible implants, as well as the resorption of the active ingredients is improved, when they are a component of a coating and/or cavity filling of an implant of biocorrodible material. More-over, the storage stability of the coated implants is improved.

This and other problems are solved by an example implant of the invention that comprises a basic body that comprises a biocorrodible, metallic material, whereby the basic body has a coating and/or a cavity filling that comprises at least one antioxidative substance or contains at least one antioxidative substance. This and other embodiments of the invention are described below.

### DETAILED DESCRIPTION

The present application claims priority on U.S. Provisional Application No. 61/231,685 filed on Aug. 6, 2009, which is incorporated herein by reference.

Some example embodiments of the invention take advantage of the discovery that the integrity of biocorrodible, metallic implants with a coating or a cavity filling that consist of at least one antioxidative substance or contain at least one oxidative substance are significantly improved which leads to longer durability of the implants at the implant site. More-over, on account of the antioxidative substance, because of its antioxidative effect, the manufactured implants have an increased shelf life.

Within the framework of the present invention, the antioxidative substances is preferably squalene although other substances will be useful in some other embodiments.

Other suitable substances include those with antioxidative effect. Some examples are: α-tocopherol (vitamin E), retinol (vitamin A), BHT (butylhydroxytoluol), BHA (butylhydroxyanisol), ascorbic acid (vitamin C), gallate such as propylgallate (E 310), octylgallate (E 311), dodecylgallate (E 312), as well as calciumdisodiummethylenediaminetetra acetate (CaNa2EDTA), carotinoides such as astaxanthin (E 161j), (β-Carotin (E 160a), canthaxanthin (E 161 g), capsanthin (E 160c), capsorubin, cryptoxanthin, lutein (E 161b), luteoxanthin, lycopene (E 160d) and zeaxanthin (E 161 h), as well as the peptide glutathione (GSH), which is produced naturally in the body, the proteins transferrin, albumin, coeruloplasmin, hemopexin and haptoglobin, as well as superoxidedismutase (SOD), glutathionperoxidase (GPX), katalase, lecithin (E 322), lactic acid (E 270), oligomers proanthocyanidine, multi phosphate as well as diphosphate (E 450), triphosphate (E 451), polyphosphate (E 452), as well as sulfur dioxide (E 220), sodium sulfite (E 221), sodium bisulfite (E 222), sodium disulfite (E 223), potassium sulfites (E 224), calcium sulfite (E 226), calciumhydrogen sulfite (E 227), potassium bisulfite (E 228) and selenium.

The listed substances can retard the oxidative influence of oxygen in air or can eliminate radicals that have already been formed. Other materials that have similar anti-oxidative functionality will likewise be useful in invention embodiments.

Squalene, also called 2,6,10,15,19,23-hexamethyl-2,6,10,14,18,22-tetracosahexaene, spinacene or supraene, belongs to the class of isoprenoids and is seen as the backbone of triterpenes (C30) and plays an important role in the biosynthesis of vitally important substances of higher organisms. This symmetrically built aliphatic compound is primarily a starting material for the formation of steroids to which important compounds such as steroles, gallic acid, steroid hormones, vitamins of the D group, saponine and cardiac glycosides belong. In the biosynthesis of, for example, cholesterol, squalene is retained intermediately by reductive dimerization of farnesyldiphosphate, which subsequently reacts again in a squalene oxide intermediate step into lanostearol, a precursor of cholesterol.

It has been discovered that squalene is a particularly suitable substance for use in some invention embodiments. An important advantage of squalene when used in invention embodiments is its significantly improved compatibility with the body with respect to toxicity compared to the other isoprenoids or isoprenoid derivatives, as well as its improved enrichment in the body. Thus, for example, lycopene and ubichinon are toxic in concentrations of 10 μmol/l. Squalene, on the other hand, is not toxic, even in concentrations of 100 μmol/l. This results in significant benefits and advantages over the prior art.

Alloys and elements are described as biocorrodible within the meaning of the invention, in which a decomposition/restructuring takes place in a physiological environment so that the part of the implant that consists of the material is entirely or at least primarily no longer present. Biocorrodible metallic materials within the meaning of the invention comprise metals and alloys selected from the group comprising iron, wolfram, zinc, molybdenum and magnesium, particularly such biocorrodible metallic materials that corrode to an alkaline product in a watery solution.

For example, the basic metallic body in some invention embodiments consists of pure iron, a biocorrodible iron alloy, a biocorrodible wolfram alloy, a biocorrodible zinc alloy or a biocorrodible molybdenum alloy. Preferably, the basic metallic body consists of or comprises magnesium. In other embodiments, the biocorrodible metallic material is a magnesium alloy. The use of biocorrodible metallic materials in implants should lead to significant decrease of rejection reactions or inflammation reactions.

A biocorrodible magnesium alloy is understood to be a metallic structure, the main component of which is magnesium. The main component is that component of the alloy that has the largest part by weight. The proportion of the main component is preferably more than 50% by weight, particularly more than 70% by weight, although other compositions will be useful (including those with less than 50%). Preferably, the biocorrodible magnesium alloy contains yttrium and other rare earth metals, as such an alloy distinguishes itself because of its physiochemical properties and high biocompatibility, particularly also its decomposition products. Especially preferred is a magnesium alloy of the following composition: rare earth metals 5.2-9.9% by weight, thereof yttrium 3.7-5.5% by weight, and the rest <1% by weight, whereby magnesium is that proportion that completes 100% by weight in the alloy. In experiments, this magnesium alloy has confirmed its special suitability in the first clinical experiments, i.e. it shows high biocompatibility, favorable processing properties, good mechanical properties and adequate corrosion behavior for many intended uses. The collective description “rare earth metals” at hand, refers to the following: scandium (21), yttrium (39), lanthan (57) and the 14 elements following lanthan (57), namely cerium (58), praseodymium (59), neodymium (60), promethium (61), samarium (62), europium (63), gadolinium (64), terbium (65), dysprosium (66), holmium (67), erbium (68), thulium (69), ytterbium (70) and lutetium (71).

The composition of the magnesium alloy is to be selected in such a way that it is biocorrodible. Within the meaning of the invention, alloys are described as biocorrodible that degrade in a physiological environment that in the end leads to a loss of the mechanical integrity of the entire implant or of the part of the implant that is made of the material. As test medium for testing the corrosive behavior of a targeted alloy, artificial plasma is used as prescribed by EN ISO 10993-15:2000 for examinations of biocorrosion (composition NaCl 6.8 g/l, CaCl₂ 0.2 g/l, KCl 0.4 g/l, MgSO₄ 0.1 g/l, NaHCO₃ 2.2 g/l, Na₂HPO₄ 0.126 g/l, NaH₂PO₄ 0.026 g/l). For this, a sample of the alloy that is to be examined is stored in a locked sample container with a specified quantity of the test medium at 37° C. At chronological intervals—adapted to the corrosive behavior that is to be expected—of a few hours up to several months—the samples are removed and examined in known ways for traces of corrosion. The artificial plasma as per EN ISO 10993-15:2000 corresponds to a medium similar to blood and thus represents a possibility of creating an environment within the meaning of the invention in a reproducible manner.

Surprisingly, it was found that an implant with a basic body that consists entirely or partially of biocorrodible magnesium alloy and a coating and/or cavity filling that consists of squalene or contains squalene, has improved resistance to corrosion. This is an important advantage over the prior art.

The positive influence of the antioxidative substance squalene achieved through invention embodiments can be explained in at least two ways. When the implants are introduced into the body, the body's own reaction is immediately initiated such as, for example, the activation of macrophages. The macrophages try to absorb the foreign substance, if such is too large, the macrophages release substances that act extremely acidic and corroding. Squalene which is a substance made by the body, prevents the immigration and activation of macrophages. A coating with this substance has the effect that the body's reaction is significantly smaller. The basic implant body is not exposed to these corrosive substances or only in a limited way. Additionally, the very lipophilic substance squalene has the effect that water reaches the implant only at a reduced speed. This slows down the normal corrosion process.

In this way, the integrity of some implants of the invention compared with the biocorrodible implants known from prior art can be extended from three weeks to two months, to nine weeks to five months. Significant cost and other savings are thereby achieved.

Implants within the meaning of the invention are devices inserted into the body in a surgical procedure and include retaining elements for bones, for example screws, plates or nails, surgical suture material, intestinal clamps, vascular clips, prosthesis in the area of hard and soft tissue and anchor elements for electrodes, particularly pacemakers or defibrillators.

Preferably, the implant is a stent. Example stents of the invention have a filigree support structure of metallic rods that are at first present for insertion into the body in non-expanded condition and which are then expanded at the site of the application and into their expanded condition. The stent can be coated onto a balloon before or after crimping.

The active coating or cavity filling can (but does not necessarily), in addition to the anti-oxidative substance, include at least one pharmaceutically active substance (active ingredient). Particularly, this pharmaceutically active substance is selected from the group including antiphlogistics, preferably dexamethasone, methylprednisolone and diclophenac; cytostatics, preferably paclitaxel, colchicine, actinomycine D and methotrexate; immunosuppressive drugs, preferably limus compounds, further preferred sirolimus (rapamycin), zotarolimus (abt-578), tacrolimus (FK-506), everolimus, biolimus, particularly biolimus A9 and pimecrolimus, cyclosporin A and mycophenolic acid; thrombocyte aggregation blocker, preferably abciximab and iloprost; statins, preferably simvastatin, mevastatin, atorvastatin, lovastatin, pitavastatin, pravastatin and fluvastatin; estrogens, preferably 17b-estradiol, daizeins and genisteins; lipid regulators, preferably fibrates; immunosuppressives; vasodilatators, preferably sartane; calcium channel blocker; calcineurine inhibitors, preferably tacrolimus; anti-inflammatory drugs, preferably imidazole; antiallergic drugs; oligonucleotides, preferably decoy-oligodesoxynucleotide (dODN); endothelial bilders, preferably fibrin; steroids; proteins/peptides; proliferation blockers; analgetics and antirheumatics; endothelial receptor antagonists, preferably bosentan; rho-kinase inhibitors, preferably fasudil; RGD peptides and cyclical RGD (cRGD) (comprising the sequence arg-gly-asp); and organic gold compounds or platinum compounds.

The example implants with at least one additional pharmaceutically active substance are marked by an increased bioavailability of the active ingredient.

The lipophilic characteristics that already have a positive effect on the corrosion behavior of implants according to the invention also have helpful effects for the distribution of active ingredients or the penetration of active ingredients. Squalene applies like a film on the interior side of the vascular structure. The active ingredient that is dissolved in the squalene film is thereby fixated longer at the implant side and has more time to penetrate the desired target tissue. Absorption by the cells is improved and with that, availability is increased.

A coating within the meaning of the invention is an application, at least in sections of the components onto the basic body of the stent. Preferably the entire surface of the basic body of the stent is covered by the coating. The thickness of the layer is preferably in the range of 1 μm to 100 μm, preferably 3 μm to 15 μm, although many other thickness ranges can be used. The coating consists of or comprises at least one antioxidative substance as well as perhaps at least one pharmaceutically active substance (in some example embodiments). Further, the coating can (but does not necessarily) contain a matrix of a biocorrodible polymer that absorbs the antioxidative substance and perhaps the pharmaceutically active substance. Alternatively, the identified substances can be components of a cavity filling contained in one or more cavities on the basic body. The one or more cavity is located at the surface or in the interior of the basic body. In some embodiments, at least one cavity is contained in an implant interior portion and isolated from the external environment by the body so that the cavity is only exposed and the release of the substances contained therein takes place only after the degradation of at least some portion of the basic body. In some other embodiments the one or more cavity may be defined by very small concave cavities on the implant outer or inner surface. The antioxidative and the pharmaceutically active substances can be present spatially separated from each other, perhaps also in various matrices in the coating.

Within the framework of some invention embodiments, the matrix is preferably a biocorrodible polymer, particularly polydioxanone; polyorthoester; polyester amide; polycaprolactone, polyglycolides; polylactides, preferably poly(l-lactide), poly(d-lactide), poly(d,l-lactide) as well as blends, co-polymers and tri-polymers of such, preferably poly(l-lactide-co-glycolide), poly(d-l-lactid-co-glycolid), poly(l-lactide-co-l-lactide), poly(l-lactid-co-trimethylene carbonate); polysaccharides, preferably chitosan, levan, hyaluronic acid, heparin, dextran, chodroitin sulfate and celluloses; polyhydroxy valerate; ethylvinyl acetate; polyethylene oxides; polyphosphoryl cholin; fibrin; albumin; and/or polyhydroxy butyric acids, preferably ataxial, isotaxial and/or syndiotaxial polyhydroxy butyric acid as well as their blends. Likewise non-degrading or slowly degrading polymers such as polyphoshazenes like polyaminophoshazenes or poly[bis(trifluoroexthoxy)phosphazene], polyurethane, such as pellethane, or polyether and polyetherblock amide, such as pebax, and polyamide.

In a preferred embodiment, the biocorrodible metallic implant has a coated basic body (on all of, or only a portion of, the basic body surface, and/or partially or completely filling one or more cavities on the body), whereby the coating and/or the cavity filling consists of an antioxidative substance.

In one example process of a method of the invention, the implant is, for example, covered with squalene using any of several suitable procedures for applying coatings. Surprisingly, it was shown that in coatings of magnesium alloys with squalene, such tend to polymerize on their own on the surface of the implant. This is an unexpected and beneficial result. After the application of squalene and storage for at least one and preferably several hours exposed to air and sunlight, a coating could be obtained that, other than pure olefin, withstands significant mechanical loads. Pure olefin consisting of squalene already increases the durability of the implant by itself, however, with respect to mechanical loads it has less stability so that the film is worn off faster, as a result of which the protection against corrosion is worsened. As a result of the polymerization initiated by the squalene film itself, the corrosion-inhibiting effect is maintained. This achieves important benefits and advantages, and represents a surprising an unexpected result.

In an additional preferred embodiment, the biocorrodible implant has a coated basic body (with the coating covering all or only a portion of the surface, and/or partially or completely filling one or more cavities on the body), whereby the coating and/or cavity filling contains at least one oxidative substance in a concentration of 1 to 20% (percent by mass in relationship to the weight of the implant), especially 2 to 10%. Other concentrations can be used in other embodiments.

In this example embodiment, the antioxidative substance reacts with the radicals that are often found at the implant site. These radicals appear at many centers of inflammation and can lead to the premature loss of the integrity of implants of the prior art in various ways. The quick neutralization of the radical species using some implant embodiments of the present invention leads to a verifiable increase in resistance against corrosion. The antioxidative effect of squalene and the other identified antioxidative substances is many times higher than, for example, for an implant coating with hyaluronic acid.

A further feature of some embodiments of the present invention concerns the use of squalene for the manufacture of an implant.

Some implants according to the invention distinguish themselves by the application of a coating consisting of or containing squalene and by a significantly improved shelf-life of the manufactured implants.

As a result of the coating in accordance with some embodiments of the invention, critical steps in production can be circumvented. The time until the implant gets into the re-packaging is no longer time-critical. Oxygen or humidity residuals in the packaging blister have a disadvantageous influence on the properties of implants of the prior art for up to one year. Embodiments of the invention thereby achieve important advantages with respect to packaging issues, shelf-live, storage stability and the like which can lead to significant cost savings.

An additional subject matter of some embodiments of the present invention concerns squalene for the prophylaxis or therapy of a restenosis or an impairment of vascular lumen in a vascular section in which a stent had been placed.

In the following, aspects of the invention are explained in more detail with examples of embodiments.

### Example of an Embodiment 1

### Coating of a Stent with Squalene as Pure Substance

A stent of biocorrodible magnesium alloy WE43 (4% yttrium by weight, 3% by weight rare earth metals except yttrium, the remainder magnesium and impurities due to the manufacturing process) is coated as follows:

The stent is cleaned of dust and residuals and clamped into a suitable apparatus for coating a stent (DES coater, developed by the company Biotronik). Using an airbrush system, the rotating stent is coated at constant conditions (room temperature; 42% ambient humidity) with squalene on one side. At a nozzle distance of 20 mm, a stent that is 18 mm long is coated after approximately 2 minutes. After the intended mass of the layer has been attained, the stent is dried for 5 minutes at room temperature before the stent is turned and again clamped in and the uncoated side is coated in the same way.

The mass of the coating applied is, for example, approximately 1-2 mg.

A stent that is manufactured in this way is subsequently crimped immediately (on a balloon, if desired) and packed air-tight and sterilized.

### Example of an Embodiment 2

### Coating of a Stent with Polymeric Squalene

Analogous to the example of an embodiment 1, a stent coated with squalene is manufactured. Subsequently, by longer exposure to air and room temperature—over 4 hours—of the coated stent, the squalene are allowed to polymerize. Other exposure times and temperatures are contemplated, with an example being at least one hour. Exposure to room temperature for periods of 4 hours, however, are believed to ensure sufficiently high rates of polymerization.

Thereupon, the stent that is manufactured in this way is crimped (onto a balloon if desired), and packaged air-tight and sterilized.

### Example of an Embodiment 3

### Coating of a Stent with Polymeric Squalene

Analogous to the example of an embodiment 1, a stent is manufactured that is coated with squalene. Deviating from example of embodiment 1, the spraying solution contains the following components:

10 ml squalene
500 μl triethanol amine
50 μl of a vinylpyrrolidon solution containing 0.3% eosin Y

Prior to coating, the components are mixed and stirred in a specified container in the dark.

The stent is coated as described in example 1, in the process the stent is exposed to a UV tube with 360 nm. With the help of the photo initiator, the cross-linking takes place directly on the stent. After 2 minutes, the stent can be turned around and be coated likewise on the other side.

### Example of an Embodiment 4

### Coating of a Stent with Polymeric Squalene

Immersion coating with cross-linked squalene.

The method contains the following components

100 parts by weight squalene
5 parts ZnO (zinc oxide)
2 parts sulfur
1,2 parts CBS (n-cyclohexyl-2-benzothiazolsulfenamide)

The solution is thoroughly stirred.

The stent is immersed into this solution, taken out and tempered for 20 minutes to 140° C. After this time has elapsed, the squalene is present in cross-linked form.

It will be apparent to those skilled in the art that numerous modifications and variations of the described examples and embodiments are possible in light of the above teaching. The disclosed examples and embodiments are presented for purposes of illustration only. Many alternatives, equivalents, and variations of elements are possible. Therefore, it is the intent to cover all such modifications and alternate embodiments as may come within the true scope of this invention.

What is claimed is:

1. Implant with a basic body that comprises a biocorrodible metallic material, the basic body having one or more of a coating and a cavity filling that comprises at least one antioxidative substance.

2. Implant according to claim 1, characterized by, that the at least one antioxidative substance is squalene.

3. Implant according to claim 1, whereby the implant is a stent.

4. Implant according to claim 1, in which the biocorrodible, metallic material is a magnesium alloy.

5. Implant according to claim 1, characterized by, that the at least one antioxidative substance is embedded into a polymeric carrier matrix.

6. Implant according to claim 1, whereby the antioxidative substance is present in a concentration between 1 to 20%.

7. Implant according to claim 6, whereby the antioxidative substance is present in a concentration of between 2 to 10%.

8. Implant according to claim 1, characterized by, that the one or more coating and the cavity filling also contains at least one pharmaceutically active substance.

9. A method for making an implant according to claim 1 and including the step of using a squalene to make the one or more coating and a cavity filling.

10. A stent having squalene for prophylaxis or therapy of a restenosis or an impairment of vascular lumen in a vascular section.

11. An implant as defined by claim 1 wherein the at least one oxidative substance comprises squalene and is provided in a coating that covers the entire surface of the implant basic body and has a thickness of between about 1 μm to 100 μm.

12. An implant as defined by claim 11 wherein:
the coating further comprises at least one pharmaceutically active material embedded with the squalene in a biocorrodible polymeric carrier matrix, the coating thickness between about 3 μm to 15 μm; and,
the implant basic body comprises a magnesium alloy including at least about 70 wt % magnesium.

13. An implant as defined by claim 1 wherein the basic body includes at least one cavity having a filling comprising the at least one antioxidative substance, and wherein the filling further comprises at least one pharmaceutically active material.

14. An implant as defined by claim 13 wherein the cavity is contained in a basic body interior and is isolated from the external environment whereby the cavity and cavity filling are only exposed after the degradation of at least a portion of the basic body.

15. An implant as defined by claim 1 wherein:
the basic body is comprised of one of pure iron, a biocorrodible iron alloy, a biocorrodible wolfram alloy, a biocorrodible zinc alloy and a biocorrodible molybdenum alloy;
the one or more of a coating and a cavity filling further comprises a pharmaceutically active material; and,
the at least one antioxidative substance is squalene.

16. An implant as defined by claim 1 wherein:
the at least one antioxidative substance is squalene in cross-linked form present in a concentration of between about 1% and 20% (wt); and
the basic body is made entirely of a magnesium alloy comprising at least 70% by weight magnesium, up to 9.9% by weight rare earth metals including yttrium.

17. An implant as defined by claim 1 wherein the basic body is comprised of a magnesium alloy including rare earth metals 5.2-9.9% by weight, thereof yttrium 3.7-5.5% by weight, and the rest <1% by weight, with magnesium accounting for the remainder of the alloy.

18. A method for making an implant as defined by claim 9 and further comprising the steps of:
providing the squalene in a concentration of between about 1% to about 20% (wt);
providing a pharmaceutically active material with the squalene wherein the one or more coating and a cavity filling further comprises the pharmaceutically active material; and
polymerizing the one or more coating and a cavity filling by exposing the implant to sunlight for a period of at least an hour.

19. A stent comprising:
a basic body comprising a biocorrodible magnesium alloy that includes at least about 5.2% rare earth metals including at least yttrium,
one or more of a coating and a cavity filling that that comprises 1% to 20% (wt %) squalene and at least one pharmaceutically active material.

20. A method for making a stent comprising:
applying one or more of a coating and cavity filling to at least a portion of a stent basic body comprised of a magnesium alloy that includes rare earth metals, the one or more of the coating and cavity filling comprising 1% to 20% (wt %) squalene and having a thickness of between about 1 μm to 100 μm, and,
polymerizing the one or more of a coating and cavity filling by exposure to air and sunlight for a period of at least 1 hour.
